SEQUENCE LISTING (15 sequences)

SEQ ID NO: 1 - protein, 7 aa, homo sapiens
  MOD_RES (3)..(3): Pro or 4Hyp
GPPGPQG

SEQ ID NO: 2 - protein, 30 aa, homo sapiens
  MOD_RES (8)..(8): Pro or 4Hyp
LQGPAGPPGEKGEPGDDGPSGAEGPPGPQG

SEQ ID NO: 3 - protein, 30 aa, bos taurus
LQGPAGPPGEKGEPGDDGPSGPDGPPGPQG

SEQ ID NO: 4 - protein, 30 aa, canis familiaris; identical to SEQ ID NO: 3
  MOD_RES (8)..(8): Pro or 4Hyp

SEQ ID NO: 5 - protein, 3 aa, homo sapiens
PQG

SEQ ID NO: 6 - protein, 5 aa, homo sapiens
PGPQG

SEQ ID NO: 7 - protein, 6 aa, homo sapiens
  MOD_RES (1)..(1): Pro or 4Hyp
PPGPQG

SEQ ID NO: 8 - protein, 30 aa, rattus sp.
  MOD_RES (8)..(8): Pro or 4Hyp
LQGPAGAPGEKGEPGDDGPSGSDGPPGPQG

SEQ ID NO: 9 - protein, 30 aa, rabbit
  MOD_RES (8)..(8): Pro or 4Hyp
LQGPAGPPGLKGEPGDSGPSGADGPPGPQG

SEQ ID NO: 10 - protein, 30 aa, mus
  MOD_RES (8)..(8): Pro or 4Hyp
LEGPAGAPGEKGEPGDDGPSGLDGPPGPQG

SEQ ID NO: 11 - protein, 30 aa, equus sp.; identical to SEQ ID NO: 3
  MOD_RES (8)..(8): Pro or 4Hyp

SEQ ID NO: 12 - protein, 7 aa, Artificial Sequence
  Synthetic Collagen I peptide fragment
GTPGPQG

SEQ ID NO: 13 - protein, 7 aa, Artificial Sequence
  Synthetic Collagen III peptide fragment
GAPGPLG

SEQ ID NO: 14 - protein, 31 aa, Artificial Sequence
  Synthetic Collagen II peptide fragment
VLQGPAGPPGEKGEPGDDGPSGAEGPPGPQG

SEQ ID NO: 15 - protein, 714 aa, homo sapiens
GPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAEGPPGPAGPAGERGEQGAPGPSGFQGLPGPPGPPGEAGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGARGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGQQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPP

(19) United States
(12) Patent Application Publication
(10) Pub. No.: US 2003/0219843 A1
(43) Pub. Date: Nov. 27, 2003

(54) Methods of diagnosing and treating abnormal growth

(76) Inventors: Dean J. Welsch, St. Peters, MO (US); Kevin L. Duffin, Manchester, MO (US); Olga V. Nemirovskiy, St. Louis, MO (US); Dawn R. Dufield, O'Fallon, MO (US); Teresa Sunyer, Wildwood, MO (US); Carol P. Howard, Fenton, MO (US); Mark Abrams, St. Louis, MO (US)

Correspondence Address: Pharmacia Corporation, Corporate Patent Department, P.O. Box 1027, Chesterfield, MO, 63006, US

(21) Appl. No.: 10/326,508

(22) Filed: Dec. 20, 2002

Related U.S. Application Data
(60) Provisional application No. 60/343,281, filed on Dec. 20, 2001.

(51) Int. Cl. G01N 33/53; G01N 33/537; G01N 33/543; C07K 16/18

(52) U.S. Cl. 435/7.92; 530/388.25

(57) ABSTRACT

A method of determining the concentration of peptides in a biological fluid resulting from the proteolytic degradation of extracellular matrix proteins for diagnosing growth disorders in vertebrates. The method includes determining the concentration of peptides resulting from the proteolytic degradation of extracellular matrix proteins in a biological fluid for determining the efficacy of drugs or agents used to treat growth disorders. A kit for determining the concentration of peptides resulting from the proteolytic degradation of extracellular matrix proteins is also disclosed.

### FIELD OF THE INVENTION

The invention relates generally to methods for identifying and quantifying peptides and, more particularly, to a method for identifying and quantifying degradation peptides resulting from enzyme cleavage of collagen. The invention also relates to the specific peptides that result from enzymatic cleavage of collagen types I, II, and III in humans and animals and the recognition of these peptides as markers in biological samples of the activity of proteolytic enzymes in diseases or physiological conditions characterized by enzymatic degradation of collagen, such as disorders involving abnormal growth and development, and the identification and quantification of the peptides to assess the efficacy of growth regulating agents and drugs used to treat or control such diseases or physiological conditions.

### BACKGROUND OF THE INVENTION

Various growth disorders affect a significant number of infants and children worldwide each year. Growth disorders may be broadly classified into two groups: those growth disorders that result in subnormal height, weight or development, and those growth disorders that result in abnormally elevated growth, weight or development.

Growth disorders may arise from a variety of causes, such as genetic predisposition, nutrition, various diseases, endocrine abnormalities, injuries, exposure to toxins, and even psychological disorders.

Treatment is most often sought for growth disorders resulting in below average height. This may be in part due to a cultural bias toward tall stature, since physical height is perceived as a desirable characteristic. Indeed, the word “stature” is synonymous with respect and preeminence in a community.

However, aberrances in growth manifested as excessive height or weight may pose significant problems to affected individuals. For example, tall subjects have greater trouble selecting automobiles in which they can comfortably sit, and may complain of low ceilings and doorways. Obese individuals may face social stresses. Both tall and obese subjects tend to be more likely to develop certain chronic disorders, such as coronary heart disease.

Sometimes, a growth disorder may be determined or suspected perinatally, either through ultrasound during pregnancy, or at birth. Intrauterine growth retardation (IUGR) may be so discerned. Infants born small for gestational age (SGA) are sometimes targeted for growth hormone therapy, or other growth promoting drug or agent administration. Frequently, a growth disorder is undiagnosed until an age is reached when it becomes apparent that the subject is developing abnormally. Measurements of somatic growth are found to lie outside of normal parameters. Such measurements may include, for example, supine length or height, weight, and head circumference. At the time of diagnosis, the subject must undergo therapy in order to approach a normal height, weight or developmental stage appropriate for the age and sex of the subject's peers. However, since the growth disorder is already manifested in an abnormal appearance, the probability of achieving a normal height, weight, or developmental stage is of dubious certainty. There is therefore a need in the art for an earlier diagnosis of growth disorders, so that a subject may receive therapy at an earlier stage, preferably before overt morphological anomalies are present.

Treatment for a given growth disorder typically involves the administration of at least one growth modulating drug or agent. A significant problem with treatment is finding an optimal dosage of growth modulating drug or agent. While there are generally accepted ranges of drug dosages, a clinician will typically administer a growth modulating drug or agent in a relatively haphazard manner, prescribing an arbitrary dosage of such drug or agent and making adjustments based upon somatic measurements representing the perceived efficacy of the drug or agent at a given dosage. This method of dosing is potentially deleterious in that a sub-optimal initial dose may further decrease the treated subject's chances of attaining the desired ultimate (adult) height, weight or development on the one hand, while an inappropriately high doses may result in unwanted side effects of the drug or agent.

Growth modulating drugs and agents may also be applied to non-human animals. Bovine somatotropin produced by recombinant microorganisms (rbSt), or extracted from pituitary gland tissue, is important commercially. It increases lactation in dairy cattle and increases size and meat production in beef cattle. It is estimated that at least 20 mg per animal per day is needed to effect commercially acceptable improvements in production. Dosing with bGH or bSt for enhancement of bovine growth is not typically optimized for an individual animal or even for a herd of genetically similar animals. Therefore, a potential inefficiency in dosing of a herd of cows or cattle may result from utilizing a set amount of growth enhancing agent or drug.

Dogs are another mammal that may benefit from growth modulating drugs or agents. In addition, dogs are sometimes used in drug safety and efficacy studies during the early stages of clinical trials.

Certain breeds of dogs, particularly retrievers, are predisposed to a condition known as canine hip dysplasia (CHD). Literally, hip dysplasia (CHD), means “badly formed hip”. In order to understand this complex problem it is first necessary to understand the anatomy of the canine hip. This ball and socket joint consists of two basic parts—the acetabulum and the femur. The femur, or thigh bone, consists of the head (the ball) and the neck (the part of the femur that joins the long shaft of the bone to the head). The acetabulum forms the socket part of the joint and it is into this socket that the head of the femur rests. A poor fit between femoral head and acetabulum is characteristic of dysplastic dogs. CHD can also be diagnosed if the femoral neck is shortened or if there is an improper angle between the femoral head and the long axis of the femoral neck.

Dogs are not born with CHD. As puppies grow, muscles and ligaments surrounding the joint become lax, and a poor fit between the bones produces excess movement of the acetabulum. The separation between the bones is called subluxation, and in severe cases, the head of the femur leaves the acetabulum. The surfaces of the bones are initially completely smooth, but in CHD the bones undergo remodeling. Bone rubbing against bone causes an irritation which results in irregular bone growth and wear on the articular surfaces. These irregular surfaces result in Osteoarthritis which can cause significant pain. As the bone of the acetabular rim is ground away, it becomes shallower and it becomes more difficult to keep the head of the femur properly seated.

CHD is a polygenic, inherited trait. It is not caused by any environmental factors, but environment can influence the expression of the disease. Among those influences are excessive weight gain or rapid growth. Unfortunately, the typically available diagnosis of CHD occurs through X-rays of the canine hip, and only after the condition has presented irreversible remodeling of the acetabulum. It would therefore be beneficial to diagnose early CHD prior to the occurrence of irreversible damage.

### SUMMARY OF THE INVENTION

It has now been discovered that a relationship exists between growth and the concentration of certain degradation products found in biological media such as urine, blood, synovial tissues, and amniotic fluid of mammals. In particular, during periods of rapid growth in immature mammals, collagen degradation products resulting from the increased turnover and remodeling of bone, cartilage and connective tissues are found in abundance as compared with those degradation products in mature mammals. Accordingly, it is believed that abnormal rates of collagen turnover are associated with abnormal levels of collagen degradation products in tissues and/or body fluids. Therefore, determining the structure and the amount of collagen degradation products in biological media is useful in the diagnosis of disorders involving abnormal growth, and is furthermore useful for monitoring the effects of growth modulating agents in the treatment of growth disorders.

The collagens are a family of 19 known distinct types of extracellular matrix proteins, divided into a fibril-forming and a non-fibril-forming group on the basis of their supramolecular complexes. Collagens are the primary fibrous protein component of skin, bone, tendon, cartilage and teeth and indeed collagen is the most abundant protein found in mammals. Of the members of the collagen family, type I collagen is the most abundant, found in connective tissues such as bone, tendon and skin. Collagen type II is found primarily in cartilage.

Generally, active and extensive collagen turnover is not considered a prominent feature in healthy adults. However, in fetal, perinatal and growing juvenile vertebrates, extensive collagen turnover is required for normal growth and development.

The breakdown of collagen type II is believed to be initiated by specific members of the matrix metalloproteinase family of enzymes, the collagenases. When collagen is degraded or cleaved by a collagenase, the cleavage takes place at a specific intra-helical site. This cleavage results in the generation of collagen fragments having an end defined by the proteolytic cleavage. For example, collagenase degradation of collagen type II results in a peptide fragment having the C-terminal sequence ending with: Gly-Pro-Xaa-Gly-Pro-Gln-Gly, where Xaa is proline or hydroxyproline. Billinghurst et al. described this primary collagenase cleavage site and developed antibodies reactive to both the carboxy-terminal and amino-terminal “neoepitopes” generated by cleavage of native human collagen type II. (J. Clin.Invest. 99:1534-1545 (1997)). Ottemess et al have described production of a monoclonal antibody directed against this carboxy-terminal “neoepitope” (Matrix Biology 18: 331-341 (1999)).

U.S. Pat. No. 6,030,792 to Otterness et. al., herein incorporated by reference in its entirety, discloses antibodies for detecting collagen fragments resulting from collagenase cleavage of type II collagen, as a method of diagnosing arthritis, and determining the efficacy of arthritis modulating pharmaceuticals. Poole et al., U.S. Pat. No. 6,132,976 herein incorporated by reference in its entirety, provides a method for evaluating cartilage degradation by immunoassay measurement of type II collagen cleavage. In any event, the prior art methods for the detection of collagen type II enzymatic cleavage by detecting the presence of peptide fragments only determine the presence of the target C-terminus portion and immediately adjacent peptide sequence using antibodies specific for these sequence regions.

One embodiment of the invention provides a method for identifying the amino acid sequence of a peptide resulting from proteolytic degradation, and quantifying the peptide in a population of normal individuals, thereby determining normal levels of the peptide. A population is selected for one or more shared characteristics, such as (but not limited to) weight, height, age and ethnicity in humans, and age, species, and breed in animals. The normal levels of each peptide thereby provide standards for use in the diagnosis of individuals suspected of having a growth disorder.

In another embodiment, the invention comprises a method of identifying and quantifying a peptide in a biological fluid sample or biological extract. In this embodiment, tandem mass spectrometric analysis is used to determine the mass of the peptide, to identify a characteristic or diagnostic carboxy-terminal amino acid sequence of the peptide upon fragmentation, and to determine the N-terminal sequence of the peptide based on additional peptide fragment ions and the observed molecular weight of the intact peptide in an individual suspected of having a growth disorder. Determination of a diagnosis of a growth disorder is aided by comparing the level of a peptide in the individual with that of a standard determined by population studies. The population can be a matched group comprising individuals selected for one or more traits shared with the individual suspected of having a growth disorder, e.g., height, weight, age, and ethnic group.

In another embodiment, an immunological assay is used for detecting the presence and/or the amount of a peptide. These assays preferably utilize an antibody directed against a collagen peptide, and more preferably against an epitope unique to a collagen peptide.

Another embodiment of the invention provides a method of detecting a peptide consisting of not more than 100 contiguous amino acids, and preferably the peptide has 7 contiguous amino acids, and more preferably, the peptide has the specific C-terminal sequence Gly-Pro-Pro-Gly-Pro-Gln-Gly (SEQ ID NO: 1) and post-translational modifications thereof. Peptide levels can be determined using mass spectroscopy, wherein fragment ions of the sequence having known mass to charge ratios are detected and quantified.

Another embodiment of the invention provides a method of detecting a peptide having the specific C-terminal sequence Gly-Pro-Pro-Gly-Pro-Gln-Gly (SEQ ID NO: 1) and post-translational modifications thereof in a biological fluid sample or biological extract to confirm the presence of peptide fragments of collagen type II that have been cleaved by a proteolytic enzyme.

Another embodiment of the invention provides a method of detecting a peptide consisting of not more than 100 contiguous amino acids, and preferably the peptide has 30 contiguous amino acids, and more preferably, the peptide has peptide sequence Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2) or post-translational modifications thereof.

Another embodiment of the invention provides a method of detecting a peptide consisting of not more than 100 contiguous amino acids, and preferably the peptide has 30 contiguous amino acids, and more preferably, the peptide has the sequence Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO: 3) and post-translational modifications thereof in a bovine biological sample, for quantification of the peptide.

Another embodiment of the invention provides a method of detecting a peptide consisting of not more than 100 contiguous amino acids, and preferably the peptide has 30 contiguous amino acids, and more preferably, the peptide is of canine origin and has the sequence leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:4) and its post-translational modifications thereof in a canine biological sample, for quantification of the peptide.

Another embodiment of the invention is the identification and quantification of SEQ ID NO:2, peptide SEQ ID NO: 3, or peptide. SEQ ID NO: 4 or derivatives or post-translational modifications thereof in a biological sample as a marker of proteolytic enzyme activity and correlation to growth.

Another embodiment of the present invention is directed to naturally occurring fragments of collagen produced by proteolytic degradation of collagen. The fragments can be identified by virtue of their sequence alignment with human collagen type II alpha 1 polypeptide as set forth in SEQ ID NO: 15. Fragments from non-human species have at least 60%, preferably, at least 75%, more preferably, at least 80%, still more preferably at least 90% and most preferably at least 95% sequence identity with human collagen type II alpha 1 polypeptide as set forth in SEQ ID NO: 15.

Another embodiment of the invention is the identification and quantification of SEQ ID NO:2, peptide SEQ ID NO: 3, or peptide SEQ ID NO: 4 or derivatives or post-translational modifications thereof in a biological sample as a marker of the presence of a disease or physiological condition in subjects characterized by the abnormal degradation of collagen type II, such disease or physiological conditions including without limitation: short stature due to growth hormone deficiency; intrautarine growth retardation (IUGR); skeletal dysplasia; neurofibromatosis; Cushing's syndrome; hypothyroidism; panhypopituitarism, including congenital panhypopituitarism and acquired panhypopituitarism; dysmorphic syndromes such as Turner, Noonan, Russell-Silver, Williams, and pseudohypothyroidism; as well as abnormally tall stature such as, for example, Klinefelter syndrome; thyrotoxosis; glucocorticoid resistence; and acromegaly.

Another embodiment of the invention is identification and quantification of peptide SEQ ID NO:2, peptide SEQ ID NO:3 or peptide SEQ ID NO:4 or derivatives or post-translational modifications thereof in a biological sample to evaluate or monitor the effectiveness of drugs or agents used to treat or control a disease or physiological condition characterized by excessive or reduced proteolytic degradation of collagen.

These and other objects, advantages and features of the invention will become apparent to those persons skilled in the art upon reading the details of the methods and the peptides fully described below.

### BRIEF DESCRIPTION OF THE DRAWINGS

FIG. 1 illustrates graphically the concentration of fragment ions of peptide corresponding in sequence to SEQ ID NO: 2 having a C-terminal sequence (SEQ ID NO: 1) present in human urine using as standard Target peptide L-leucyl-L-glutaminylglycyl-L-prolyl-L-alanylglycyl-L-prolyl-L-prolylglycyl-L-alpha-glutamyl-L-lysylglycyl-L-alpha-glutamyl-4-hydroxy-L-prolylglycyl-L-alpha-aspartyl-L-alpha-aspartylglycyl-L-prolyl-L-serylglycyl-L-alanyl-L-alpha-glutamylglycyl-L-prolyl-4-hydroxy-L-prolylglycyl-L-prolyl-L-glutaminylglycine, correlated by age;

FIG. 2 illustrates graphically the concentration of fragment ions of peptide corresponding in sequence to SEQ ID NO: 2 having a C-terminal sequence (SEQ ID NO: 1) present in adult human urine using SEQ ID NO: 2, produced by Elisa using as standard Target peptide L-leucyl-L-glutaminylglycyl-L-prolyl-L-alanylglycyl-L-prolyl-L-prolylglycyl-L-alpha-glutamyl-L-lysylglycyl-L-alpha-glutamyl-4-hydroxy-L-prolylglycyl-L-alpha-aspartyl-L-alpha-aspartylglycyl-L-prolyl-L-serylglycyl-L-alanyl-L-alpha-glutamylglycyl-L-prolyl-4-hydroxy-L-prolylglycyl-L-prolyl-L-glutaminylglycine of urine from normal adult human subjects, and adult subjects medically diagnosed with and exhibiting signs and symptoms of osteoarthritis;

FIG. 3 illustrates the fragment ions of peptide SEQ ID NO: 3, having a C-terminal sequence (SEQ ID NO: 1) produced by tandem mass spectrometric analysis of urine from a bovine subject exhibiting signs and symptoms of arthritis;

FIG. 4 illustrates the fragment ions of a peptide SEQ ID NO: 2, produced by tandem mass spectrometric analysis of urine from a human subject;

FIG. 5 illustrates the fragment ions of a peptide SEQ ID NO: 4, produced by tandem mass spectrometric analysis of urine from a canine subject;

FIG. 6 illustrates the fragment ions of a human collagen type II synthetic peptide standard produced by tandem mass spectrometric analysis;

FIG. 7 is a comparison of the extracted ion chromatograms for the collagen type II target peptide SEQ ID NO: 2 determined by tandem mass spectrometric quantification of urine from a human subject that did not exhibit signs and symptoms of arthritis (bottom), from urine of a human subject that was medically diagnosed with and exhibited signs and symptoms of osteoarthritis (middle), and from urine of a human subject that did not exhibit signs and symptoms of arthritis that was spiked with a synthetic standard of the collagen II peptide;

FIG. 8 shows the standard curve of the human collagen type II synthetic peptide spiked into normal human urine at concentrations ranging from 30 pg/mL to 100 ng/mL;

FIG. 9 illustrates the fragment ions of a synthetic collagen type I peptide SEQ ID NO: 12 produced by tandem mass spectrometry; and

FIG. 10 illustrates the fragment ions of a synthetic collagen type III peptide SEQ ID NO: 13 produced by tandem mass spectrometry.

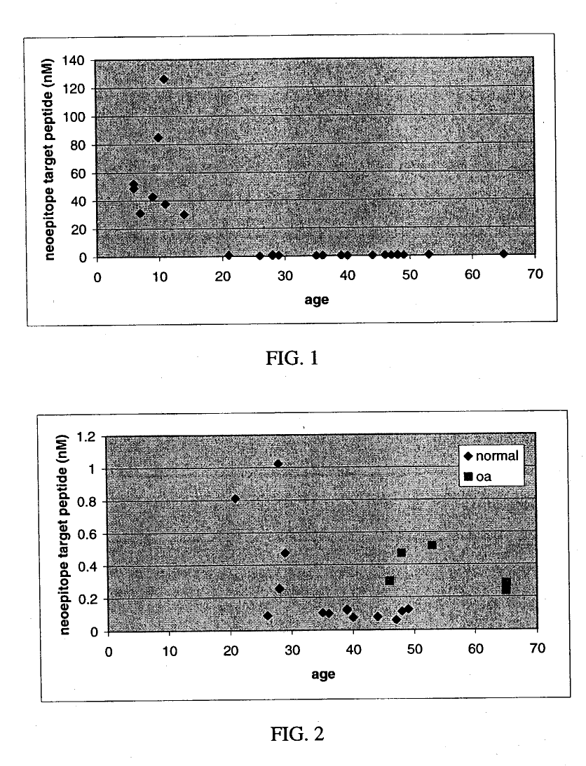

### DETAILED DESCRIPTION OF THE INVENTION

The present invention is directed to the identification and quantification of collagen peptides, particularly in biological samples from humans or animals. In one embodiment, the structure and quantity of collagen peptides are determined for a population of normally growing humans or animals, wherein each population is defined by one or more common traits, such as (but not limited to) age, weight, gender, and ethnicity in humans, and age, weight, species, and breed in animals for the purpose of statistical grouping of the quantification of peptides found in such humans or animals to establish a normal distribution of collagen peptide for use as a comparative standard.

In another embodiment of the present invention, the presence and/or quantity of a peptide in a biological sample is determined, and compared with a standard to diagnose or assist in the diagnosis of a growth disorder in the subject.

In another embodiment of the present invention, the efficacy of a growth modulating drug or agent is assessed by quantifying the peptide content in samples obtained before and after administration of the growth modulating drug or agent. A change in peptide content from an abnormal level towards a more normal level is expected if the growth modulating drug or agent is effective. This approach can be used both experimentally in the development of new growth modulating drugs or agents, or in connection with therapy to determine if the administration of a therapeutic agent such as a growth modulating drug or agent is effective in the treatment of a growth disorder.

In another embodiment of the present invention, the efficacy of a drug or agent not previously indicated in or approved for the therapeutic treatment of a growth disorder is determined, at least in part, from quantification of a collagen peptide in a time course study wherein repeated quantifications of collagen peptide from a subject is performed over an appropriate interval of time.

These and other variations will occur to those skilled in the art in light of the following description and examples.

Definitions

A “growth modulating drug or agent” or “growth regulating drug or agent” means a compound, composition of matter, pharmaceutical, chemical, or combinations thereof, administered for the purpose of appropriately increasing or decreasing the rate or ultimate attainment of height, weight, or morphologic proportion.

Growth modulating drugs and agents for promoting or enhancing normal growth (also referred to as growth upregulating drugs or agents) include both naturally occurring and synthetic drugs and agents. A non-limitative list of growth upregulating drugs includes: growth hormones and growth hormone secretagogues such as, for example, SM 130686 (Sumitomo) capromorelin (Pfizer), mecasermin (Fujisawa), sermorelin (Salk Institute, Bio-Technology General), somatrem, somatropin (Novo Nordisk; Yamanouchi), somatomedin (C Llorente; Pharmacia Corporation) examorelin, tabimorelin; CP 464709 (Pfizer), LY 426410 and LY 444711 (Lilly); human growth hormone fusion proteins such as ALBUTROPIN; polyethylene glycol growth hormones such as the cysteine-pegylated growth hormone, BT 005 (Bolder BioTechnology Inc.); growth hormone releasing factors such as ThGRF 1-44 (Theratechnologies); insulin-like growth factor-1 (IGF-1 or somatomedin C) and IGF-1 secretagogues; insulin-like growth factor-2 (IGF-2 or somatomedin A) and IGF-2 secretagogues; Growth hormone releasing factors such as L 165166 (Merck & Company); growth hormone gene therapy; and compounds which inhibit fibroblast growth factor receptor-3 (FGFR-3) tyrosine kinase.

Growth modulating drugs and agents for retarding excessive growth include both naturally occurring as well as synthetic drugs and agents, such as, by way of example and without limitation: somatostatin and somatostatin analogues such as lanreotide (Beaufour Ipsen), octreotide (Novartis); and human growth hormone receptor antagonists such as pegvisomant (Sensus; Pharmacia Corporation).

A “growth disorder” means a condition in-which an immature subject fails to attain a height, weight, proportional morphology, or any combination thereof within accepted normal parameters for a given age group. Growth disorders are intended to describe both less than optimal growth, such as, for example short stature, as well as excessive growth, such as, for example, acromegaly.

“Growth disorders” resulting in abnormally low weight or short stature include without limitation: chronic renal insufficiency; growth hormone deficiency; genetic abnormalities such as, for example dysmorphic syndromes such as Turner's syndrome, Noonan syndrome, Russell-Silver disorder, William's syndrome, Cushing's syndrome; hypothyroidism; panhypopituitarism, including congenital panhypopituitarism and acquired panhypopituitarism; and pseudohypothyroidism.

“Growth disorders” characterized by abnormally abundant growth include, without limitation, disorders such as, for example, Klinefelter syndrome; thyrotoxosis; glucocorticoid resistence; and acromegaly.

“Normal growth,” or “growth typical for age” mean a value determined to fall within an average, weighted average, median, mean or other statistical measurement of a population of infants or children that are considered to be without a growth disorder. The value ascribed to “Normal growth,” or “growth typical for age” may vary for a given sex, ethnic group or geographic region.

“Biological Media” or “Biological sample” means any biological fluid that might contain the collagen degradation products and be of interest to assay by this procedure. These include: blood, synovial fluid, urine, spinal fluid, bronchiolar lavage fluid, lymph, the vitreous humor of the eye, extracts of tissues, tissue culture supematants, extracts of cartilage, etc., Biological media need not be limited to human samples, but may also be obtained from a similar variety of animal media (mouse, rat, hamster, guinea pig, dog and bovine, for example) in a fashion similar to the examples above.

“Immunoassay” means an assay for a substance (complex biological such as a protein or a simple chemical) based on using the binding properties of antibody to recognize the substance which may be a specific molecule or set of homologous molecules. The assay may involve one or more antibodies.

“Direct Assay” means a process in which the antibody binds directly to an antigen such as in a biological specimen (cells, tissues, histological section, etc.) or to antigen adsorbed or chemically coupled to a solid surface. The antibody itself may be labeled to enable the determination of the amount of antibody bound to the antigen. Alternatively, the antibody (now termed primary antibody) is detected with a secondary labeled antibody that will demonstrate that binding of the primary antibody had occurred.

“Competitive Assay” means an assay based on the binding properties of a single antibody molecule. Typically, a labeled antigen is used to compete with an unknown antigen and the amount of unknown antigen is determined in terms of how much of the labeled antigen is displaced by the unknown antigen. The label may be radioactive, optical, enzymatic, florescent polarizing, florescent quenching, or other label. The antibody may be monospecific or bispecific.

“Sandwich Assay” means a double antibody assay in which both antibodies bind to the antigen, forming a trimeric immune complex or sandwich containing the two antibodies with the antigen between them. One antibody is utilized to localize the immune complex to the detection surface or chamber. This antibody is termed the capture antibody. The other antibody bears a label that will allow the immune complex to be detected. It is called the detection antibody. If an immune complex is not formed (no antigen is present), then the capture antibody is unable to bring the detection antibody to the detector. If antigen is present, then an immune complex will form and the capture antibody will be joined with the detection antibody such that the amount of detection antibody in the immune complex is quantitatively related to the amount of antigen present.

The invention includes a method of determining the presence of, and identifying the structure of, peptide degradation products of specific collagenase enzyme activity.

The present invention also includes the detection of naturally occurring collagen polypeptide fragments in body fluids and tissues for the purpose of detection of disorders. As used herein, the term “detection” is intended to the determining of the presence of a disorder in a patient, the distinguishing of the disorder from other diseases, the estimation of prognosis in terms of probable outcome of the disorder and prospect for recovery, the monitoring of the disorder status or the recurrence of the disorder, the determining of a preferred therapeutic regimen for the patient and the targeting of therapy. Such detection can involve any method known in the art for detecting proteins can be used including, but are not limited to immunodiffusion, immunoelectrophoresis, immunochemical methods, binder-ligand assays, immunohistochemical techniques, agglutination and complement assays. (for example see Basic and Clinical Immunology, Sites and Terr, eds., Appleton & Lange, Norwalk, Conn. pp. 217-262, 1991 which is incorporated by reference). Preferred are binder-ligand immunoassay methods including reacting antibodies with an epitope or epitopes of the collagen fragment polypeptides and competitively displacing a labeled a collagen fragment or a derivative thereof.

The term “subject,” as used herein, refers to both humans and animals.

As used herein, a derivative of a collagen fragment polypeptide is intended to include a polypeptide in which certain amino acids have been deleted or replaced or changed to modified or unusual amino acids wherein the collagen fragment polypeptide derivative is biologically equivalent to collagen fragment polypeptide and wherein the polypeptide derivative cross-reacts with antibodies raised against the collagen fragment polypeptide. By cross-reaction it is meant that an antibody reacts with an antigen other than the one that induced its formation.

Kits for measuring the levels of a collagen fragment polypeptide in patient samples are also within the scope of the present invention. Such assay kits can be based any known protein assay method such as immunodiffusion, immunoelectrophoresis, immunochemical methods, binder-ligand assays, immunohistochemical techniques, agglutination and complement assays. Included in such kits are suitable reagents for conducting the assays.

Antibodies to a collagen fragment polypeptide are also within the scope of the present invention. Such antibodies can be polyclonal or monoclonal antibodies. Polyclonal antibodies can be prepared by immunizing rabbits or other animals by injecting antigen followed by subsequent boosts at appropriate intervals. The animals are bled and sera assayed against a purified collagen fragment polypeptide. When using avian species, e.g. chicken, turkey and the like, the antibody can be isolated from the yolk of the egg. Monoclonal antibodies can prepared after the method of Milstein and Kohler by fusing splenocytes from immunized mice with continuously replicating tumor cells such as myeloma or lymphoma cells. (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

Specific antibodies, either polyclonal or monoclonal, to a collagen fragment polypeptide can be produced by any suitable method known in the art as discussed above. For example, murine or human monoclonal antibodies can be produced by hybridoma technology or, alternatively, the collagen fragment polypeptide, or an immunologically active fragment thereof, or an anti-idiotypic antibody, or fragment thereof can be administered to an animal to elicit the production of antibodies capable of recognizing and binding to collagen fragment polypeptides. Such antibodies can be from any class of antibodies including, but not limited to IgG, IgA, IgM, IgD, and IgE or in the case of avian species, IgY and from any subclass of antibodies.

The present invention can be used to detect cleavage by proteolytic enzymes, such as matrix metalloproteinases-1, -8, and -13, that results in peptides with C-terminal amino acid sequences having characteristic fragment ions upon collisional activation that can be identified by the methods of one embodiment of the present invention. An embodiment of the present invention allows the diagnosis and prognosis of physiological conditions characterized by cartilage degradation through the identification and quantification of collagen degradation peptides discovered to be present in biological samples of subjects exhibiting signs and symptoms of diseases characterized by abnormal cartilage degradation, such as in, for instance, short stature and acromegaly. The invention is particularly useful in that it allows the detection and quantification of post-translational analogs of the peptides.

One method of the invention relates to mass spectrometric analysis of peptide fragments in biological fluid samples or biological extracts to detect and measure protein degradation products, particularly proteins degraded by proteolytic enzymes. The proteolytic enzyme cleavage of a protein yields degradation peptides having a characteristic C-terminal amino sequence, depending upon the species, the source of the protein, and the involved enzyme. In one embodiment of the invention collagen degradation peptides of a known mass in a biological sample are separated by chromatographic techniques and then fragmented by collisional activation in the mass spectrometer using techniques known in the art. Upon collisional activation, the peptides yield fragments having characteristic mass to charge ratios. By detecting the presence of the characteristic peptide fragments of the C-terminus, for example, the sequence of the C-terminus is confirmed. Confirming the C-terminus sequence and the mass of the degradation peptide allows the deduction of the N-terminus of the peptide.

The peptide and fragment ion molecular weights determined in the tandem mass spectrum of the peptide are compared to those expected from known protein sequences, with postulated post-translational modifications, found in a protein database to determine the entire amino acid sequence including post-translational modifications of the peptide. The identification methods of the present invention allow identification of the C-terminal and the N-terminal sequences and, therefore, the amino acid sequence of the entire degradation peptide, including potential hydroxylation of proline amino acids present in the peptide. The peptide identity is further validated by synthesizing the peptide and demonstrating that the synthetic peptide provides the same analytical data, including liquid chromatographic elution time and mass spectrometric fragmentation as the peptide identified from biological sample. Once the peptide is identified, a standard peptide with the same or similar sequence can be used to determine the relative amount of the enzymatically cleaved peptide in the biological sample. This quantification can be used to assess proteolytic enzyme activity, for diagnosis or prognosis of diseases, and to evaluate drugs or agents used modulate growth. Drugs or agents used to modulate growth include, without limitation, drugs or agents administered to increase growth, such as human growth hormone, human growth hormone analogs, and insulin-like growth factor-1, as well as drugs or agents administered to retard growth such as pegvisomant.

One embodiment of the invention provides for the identification and quantification of collagen cleavage products. For example, one embodiment of the invention provides for the identification and quantification of collagen types I, II and III breakdown products. A preferred embodiment of the present invention provides detection and quantification of specific, newly discovered protein cleavage products of collagen type II in a biological sample. The invention can be used to detect the presence of proteolytic enzyme degradation products resulting from the degradation of collagen type II by enzymes, such as matrix metalloproteinases, particularly matrix metalloproteinases-1, -8, and -13.

Identification and quantification of collagen type II peptide fragments in urine samples of humans indicate that post-translationally modified analog peptides of Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2), wherein position 14 and 26 are 4-hydroxyproline, are present in detectable amounts in human subjects medically diagnosed with and displaying signs and symptoms of a growth disorder. Identification of collagen type II peptide fragments in urine samples of cattle indicate that post-translationally modified analog peptides of Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:3), wherein position 14 and 26 are 4-hydroxyproline, are present in detectable amounts in bovine subjects displaying signs and symptoms of a growth disorder.

Likewise, identification and quantification of collagen type II peptide fragments in urine samples of dogs indicate post-translationally-modified analog peptides of Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:4), wherein position 14 or 26 is 4-hydroxyproline, are present in detectable amounts in canine subjects displaying signs and symptoms of a growth disorder. It is understood that the significant post-translational modifications of SEQ ID NO:2, SEQ ID NO: 3 and SEQ ID NO: 4 include the post-translational modification of proline to hydroxyproline at positions 8, 14 and/or 16 and possibly of lysine to hydroxylysine at position 11. The post-translational modifications appear in varying levels in different species. The originally translated peptides themselves, as well as permutations of the post-translational modifications, may be found in urine of subjects and are included among the embodiments of the invention.

It is believed these degradation peptides are a result of cleavage of collagen type II by collagenase, particularly matrix metalloproteinases, and more particularly matrix metalloproteinase-13 (MMP-13). Thus, the peptides SEQ ID NO:2, SEQ ID NO: 3 or SEQ ID NO:4, and particularly the post-translational analogs thereof, found in biological samples function as markers of enzyme activity and/or markers of diseases or conditions characterized by abnormal collagen type II breakdown found in a growth disorder. Identifiable derivatives or modifications of the peptides also can function as markers and are included within the scope of the invention.

The “C-terminal sequence” collagen type II alpha 1 polypeptide has a sequence of 7 amino acids (SEQ ID NO: 1) extending from Gly-469 to Gly-475 of collagen type II alpha 1. Although the human polypeptide is identified as a portion of human collagen type II alpha 1 polypeptide, it is intended that the term “C-terminal sequence polypeptide” include orthologous sequences from non-human mammalian species as well as from non-mammalian vertebrate species. The sequences of such orthologous molecules can be readily determined by alignment of orthologous plasminogen sequences with human collagen type II alpha 1 polypeptide. Alternatively, putative orthologous C-terminal sequence polypeptide molecules can be aligned with human C-terminal sequence polypeptide molecules. It is believed that non-human mammalian orthologous C-terminal sequence polypeptides will have at least 75% sequence identity, i.e. at least 75% identical amino acids, as compared to human C-terminal sequence and that non-mammalian vertebrate orthologous C-terminal sequence polypeptides will have at least 60% sequence identity as compared to human C-terminal sequence polypeptide.

The peptide of collagen type II alpha 1 polypeptide has a sequence of 30 amino acids as set forth in SEQ ID NO:2, extending from Lys-446 to Gly-475 of collagen type II alpha 1. Although the human polypeptide is identified as a portion of human collagen type II alpha 1 polypeptide, it is intended that the reference to this peptide include orthologous sequences from non-human mammalian species as well as from non-mammalian vertebrate species. The sequences of such orthologous molecules can be readily determined by alignment of orthologous plasminogen sequences with human collagen type II alpha 1 polypeptide. Alternatively, putative orthologous molecules can be aligned with human polypeptide molecule having a sequence as set forth in SEQ ID NO: 2. It is believed that non-human mammalian polypeptide orthologous to human polypeptide having a sequence as set forth in SEQ ID NO: 2 will have at least 75% sequence identity, i.e. at least 75% identical amino acids, as compared to the human polypeptide sequence as set forth in SEQ ID NO:2 and that non-mammalian vertebrate orthologous polypeptides will have at least 60% sequence identity as compared to human polypeptide sequence as set forth in SEQ ID NO:2 Methods of sequence alignment for identifying homologous sequences which can be either paralogs or orthologs are well known in the art. For example, two or more sequences can be aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NBRF, Washington, Vol. 5, suppl. 3, p. 345, 1978).

The identification and quantification of the peptides SEQ ID NO:2, SEQ ID NO:3 or SEQ ID NO:4, and their post-translationally modified analog peptides, in general, is performed by the following illustrative analytical detection method:

A biological sample, such as urine, plasma, blood, amniotic fluid or synovial fluid is collected from the subject.

The concentration of degradation products of collagen is measured. In one embodiment, the relative molecular mass of abundant peptides in the sample is determined by a first stage mass spectrometry and then the peptides are fragmented and fragment ions analyzed by second stage of the tandem mass spectrometer, as will be further explained below. By way of example, the post-translational analogs of peptides SEQ ID NO: 2, SEQ ID NO:3 and SEQ ID NO: 4 found in picomolar to nanomolar amounts in the biological samples of subjects exhibiting signs and symptoms of a growth disorder have a mass/charge ratio in the range of approximately 900 to approximately 1000 with known variations of the mass/charge ratio within this range depending upon the post-translational modifications of proline and in some examples, lysine. The charge for these peptides is +3. As will be discussed more specifically in the examples below, the relative mass/charge of the abundant post-translation analog of peptide SEQ ID NO: 2 was determined to be 914.4 and the charge was +3. The mass/charge of the abundant post-translation analog of peptide SEQ ID NO: 3 was shown to be 918.7 and, the relative mass/charge of the abundant post-translation analog of peptide SEQ ID NO: 4 was shown to be 913.4, as described in the examples below.

The peptides can be separated from the biological matrix components of the sample by an appropriate separation method known to the art. For example, chromatographic or electrophoretic separation can be used in this step. Other appropriate separation or “clean up” methods are contemplated by the invention. In liquid chromatographic separation the eluant containing the peptides of the target mass are introduced to a mass spectrometer through an appropriate liquid chromatography/mass spectrometry interface.

The peptides are fragmented by collisional activation by a neutral gas in a collision cell using collision energies and methods known to the art resulting in collision-induced dissociation of the peptide to create corresponding fragment ions. The spectrum of the fragment ions is analyzed. Peptides having the C-terminal sequence Gly-Pro-Pro-Gly-Pro-Gln-Gly (SEQ ID NO: 1), where proline at the third position may be in the post-translationally modified form, hydroxyproline, fragment to yield product ions having characteristic mass to charge ratios. As demonstrated, where the proline at position 3 of SEQ ID NO: 1 is 4-hydroxyproline, the respective masses of the characteristic fragments are approximately m/z 301, 471 and 568. These characteristic fragments of C-terminal sequences are a result of the dominant cleavage of the peptide bonds on the N-terminal side of proline. The peptides having a C-terminus of Gly-Pro-Pro-Gly-Pro-Gln-Gly (SEQ ID NO: 1), i.e., the non-modified form, will fragment to form fragment ions of the sequence Pro-Gln-Gly (SEQ ID NO: 5) having a mass of approximately 301, sequence Pro-Gly-Pro-Gln-Gly (SEQ ID NO: 6) having a mass of approximately 455 and sequence Pro-Pro-Gly-Pro-Gln-Gly (SEQ ID NO: 7) having a mass of approximately 552.

Identification of the characteristic product ions is a strong indication of the presence of the C-terminal sequence Gly-Pro-Pro-Gly-Pro-Gln-Gly (SEQ ID NO: 1) or post-translation modifications thereof.

As described below, through this method of identification of peptide fragments in biological samples it was discovered that in human subjects medically diagnosed and exhibiting arthritic signs and symptoms, the predominant degradation peptide is the post-translational modification of Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2), where prolines at positions 14 and 26 are 4-hydroxyproline. The post-translationally-modified peptides SEQ ID NO: 3 and SEQ ID NO: 4 were discovered to be the predorminant degradation peptides in cattle and dogs, respectively, which also exhibited signs and symptoms of abnormal cartilage degradation characteristic in a growth disorder.

It will be noted that when any proteolytic enzyme cleaves a protein or peptide, there is a resulting characteristic C-terminus and potential post-translational modifications thereof. This is due to the fact that specific enzymes cleave proteins at specific sites in the amino acid sequence. Fragmentation of a peptide having a specific C-terminus will yield characteristic fragments upon collisional activation having specific mass to charge ratios. That is, upon fragmentation each C-terminus will have its own fingerprint. Consequently, the described method can be used to determine the amino acid sequence of any peptide that results from proteolytic cleavage leaving a known C-terminus that fragments into characteristic product ions.

The method of the present invention is not limited to identification and quantification of peptides resulting from the degradation of type II collagen or the specific peptides described herein by sequence identification numbers. The analytical methods of the present invention can be used to detect the presence of proteolytic enzyme degradation products resulting from the degradation of proteins other than the illustrated collagen type II or by enzymes other than collagenases, specifically, metalloproteinase-13 (MMP-13). By way of example only, the method of the present invention can be used to identify proteolytic degradation products resulting from cleavage by other matrix metalloproteinases such as matrix-metalloproteinase-1 (MMP-1) and matrix metalloproteinase-8 (MMP-8).

The following examples are illustrative, and not intended to limit the scope of the invention in any way. Numerous variations will occur to those skilled in the art, and these variations are within the intended scope of the invention.

### EXAMPLE 1

A. Selection of a First Monoclonal Antibody

A first monoclonal antibody to be used as a detection antibody is isolated or selected from known, available monoclonal antibodies such that the monoclonal antibody demonstrates binding to a particular degradation product (a Type II collagen fragment, for example) but no significant binding to intact collagen. The generation and characterization of such a monoclonal antibody is well known in the art.

Monoclonal antibodies can be of any class of antibody, e.g., IgG, IgA, IgD, IgE, and IgM.

B. Selection of a Second Monoclonal Antibody

A second monoclonal antibody to be used as a capture antibody is isolated or selected from known, available monoclonal antibodies such that the monoclonal antibody demonstrates binding to degradation products (collagen fragments) but no significant binding to intact collagen. The generation and characterization of such a monoclonal antibody is well known in the art.

C. Sandwich Assay Using a Capture Antibody (CAb) and a Monoclonal Antibody as the Detection Antibody (Dab) for Determining Concentration of a Degradation Product in a Biological Sample.

The second monoclonal antibody of part B, above (the capture antibody, CAb) is added to Nunc Maxisorp (VWR, Boston, Mass.) 96-well plates with CAb at 10.mu.g/mL in 0.05M sodium borate buffer, pH 8.5 using 100.mu.L/well (except for control wells numbered 4, 5 and 6, see Table 1) and incubated for 18-48 hours at 4.degree. C.

The plate is washed three times with DPBS with 0.05% TW-20 (Sigma), (DPBS/TW-20); 200.mu.L/well may be used.

Wells in the plate are blocked with 1% non-fat dry milk (NFDM) dissolved in DPBS (NFDM DPBS) prepared freshly, i.e., held on ice for no more than the day of use, using 100.mu.L/well incubated for 1 hour at RT.

The blocking solution is discarded, the wells rinsed one time with 200.mu.L of DPBS/TW-20.

A peptide, for example corresponding in amino acid sequence to SEQ ID NO 2, is diluted in 0.1% NFDM DPBS to concentrations shown in Table 1. The exemplary peptide has the sequence L-leucyl-L-glutaminylglycyl-L-prolyl-L-alanylglycyl-L-prolyI-L-prolylglycyl-L-alpha-glutamyl-L-lysylglycyl-L-alpha-glutamyl-4-hydroxy-L-prolylglycyl-L-alpha-aspartyl-L-alpha-aspartylglycyl-L-prolyl-L-serylglycyl-L-alanyl-L-alpha-glutamyl glycyl-L-prolyl-4-hydroxy-L-prolylglycyl-L-prolyl-L-glutaminylglycine, as set forth in the Sequence Listings as SEQID NO: 2 and may be synthesized and purified by any custom protein synthesis laboratory, such as Anaspec Inc (San Jose, Calif.), for example.

The dilutions of this exemplary peptide of SEQ ID NO: 2, the samples at appropriate dilutions, and the controls are placed into the specified wells of the microliter plate as shown in Table 1.

TABLE 1
Outline of a microtiter plate and antibody coating scheme
Peptide (SEQ ID NO 2) ng/mL

| peptide | peptide | peptide | peptide | peptide | peptide | peptide | peptide | peptide | peptide | peptide | peptide |
|---|---|---|---|---|---|---|---|---|---|---|---|
| 2 | 1.33 | 0.889 | 0.59 | 0.4 | 0.26 | 0.18 | 0.12 | 0.08 | 0.05 | 0.03 | 0.02_{—} |
| ″ | ″ | ″ | ″ | ″ | ″ | ″ | ″ | ″ | ″ | ″ | ″ |
| sample | sample | sample | sample | sample | sample | sample | sample | sample | sample | sample | sample |
| ″ | ″ | ″ | ″ | ″ | ″ | ″ | ″ | ″ | ″ | ″ | ″ |
| ″ | ″ | ″ | ″ | ″ | ″ | ″ | ″ | ″ | ″ | ″ | ″ |
| ″ | ″ | ″ | ″ | ″ | ″ | ″ | ″ | ″ | ″ | ″ | ″ |
| ″ | ″ | ″ | ″ | ″ | ″ | ″ | ″ | ″ | ″ | ″ | ″ |
| controls | controls | controls | controls | controls | controls | controls | controls | controls | controls | controls | controls |
| 1 | 1 | 2 | 2 | 3 | 3 | 4 | 4 | 5 | 5 | 6 | 6 |

TABLE 2
Additions to the control wells

| Controls: | | | Biotinylated | Anti-biotin antibody |
|---|---|---|---|---|
| CAb | | peptide | DAb | HRP-labelled |
| 1 | + | − | − | + |
| 2 | + | + | − | + |
| 3 | + | − | + | + |
| 4 | − | + | + | + |
| 5 | − | + | − | + |
| 6 | − | − | + | + |

The wells are washed three times with 200 μ.L/well of DPBS TW-20.

Biotin-conjugated DAb (Bt-DAb) is added to all peptide of SEQ ID NO: 2 containing wells, all sample wells, and all control wells except 1,2, and 5. Bt-DAb (100 μL/well) at 1 μg/mL in 0.1% NFDM DPBS is added to each well and the plate is incubated for 40 min at 37.degree. C.

Optionally, the DAb may be biotinylated using 37.mu.g of biotin-N-hydroxysuccinamide (Pierce Chemical) per mg of DAb for 2 hrs and then dialyzed overnight using a 10 kD cut-off dialysis cassette (Pierce Chemical).

The wells are washed three times with 200.mu.l/well of DPBS TW-20.

Mouse monoclonal anti-biotin antibody conjugated with HRP (available from Jackson ImmunoResearch) is diluted 1/5000 in 0.1% NFDM DPBS and 100 mu.L/well is added to all wells and incubated for 30 minutes at RT.

The wells are washed three times with 200.mu.L/well with DPBS TW-20. 100.mu.L/well of I-step Turbo (ready to use 3,3′,5,5′-tetramethyl benzidine; Pierce Chemical) is added to each well and incubated at RT for approximately 10 minutes. Color development may be stopped with 2N H₂ SO₄. The results are read on a spectrophotometer at 450 nm.

From the concentrations of a peptide of SEQ. ID NO. 2 and the resulting optical density reading at 450 nm, a standard curve can be constructed. Over the linear portion of the curve, a regression line is used to fit the data. For samples that fall outside of the linear portion, the concentrations can be read off the graph or the samples may be diluted to fall within the standard portion of the curve, or they may be below the limit of detection. The regression between log (nM) and OD450 gives a slope OD450/log(nM) and an intercept OD450. When unknown samples are run, the calibration curve can be used to determine of concentration of collagenase-generated type II collagen fragments from the optical density of the sample. The following calculation can be used to determine the concentration of the degradation fragment in a biological sample:

Log(Concentration[nM])=(Sample OD450−Intercept)/Slope

Thus, the inverse log of the resulting number provides the concentration in nM. Other appropriate peptides or collagen fragments can be substituted to prepare a standard curve. The units are expressed in terms of molar equivalents of standard. In this case, the units are nM equivalents of peptide of SEQ ID NO: 2.

D. Comparison of Sample Concentration to Known Standard Concentration

The concentration calculated from the biological sample is compared with a standard. The standard is preferably provided as adjusted based upon various factors, including without limitation: sex; weight; height; or a combination thereof, for a statistically normal growth degradation product concentration at a given age. Thus, for example, a statistically normal male eight year old may have a concentration of about 42 nM of a degradation product comprising a peptide corresponding to SEQ ID NO 2. A standard error is taken into account for the approximate standard concentration. Values outside of the standard error are considered to be abnormal. Thus, for example, if a sample taken from an eight year old is found to contain 22 nM of the degradation product, and the normal concentration is predicted to be 42 nM±8 nM, the sample is considered to represent an abnormal concentration, (at least 12 nM [35%] under the standard concentration) indicating a need for a growth upregulating drug or agent.

E. Therapeutic Dosing Based Upon Comparative Sample and Standard Data

The concentration ascertained from the sample may be used to establish an appropriate dose of growth modulating drug or agent. For example, a deficiency of 12 nM in an eight year old male subject may indicate an initial dose of 0.03 mg/kg per day of growth hormone. Other growth upregulating drugs or agents may be initially dosed proportionally as well. Optionally, a measure of other physiological factors, such as, for example, endogenous growth hormone, may be obtained to further assess a growth disorder, or establish a dosing regimen.

F. Determination of Efficacy of Treatment

In addition, the methods of Example 1, parts C and D may be repeated at predetermined intervals, in order to ascertain the relative success of the treatment method selected. For example, at two months following initial diagnosis and commencement of treatment, a second sample may be collected and compared to a standard. This comparison will show a relative increase in degradation products, correlating to an increase in cartilage, connective tissue or bone growth. As the biological sample approaches the normal standard, dosing may be modified so as to customize the dose administered to the subject, and thereby optimize treatment efficacy and potentially reduce unwanted side effects and the expense of an inappropriately high dosage of growth modulating drugs.

### EXAMPLE 2

### Identification of a Bovine Collagen Type II Peptide Fragment

The following procedures are employed to determine that the post-translationally modified peptide of SEQ ID NO: 3 is a peptide in the urine of cows:

Urine is collected from the subject.

pH of the urine is adjusted to 7.1 with ammonium acetate

Sample was fractionated using a mixed-mode ion exchange reversed phase preparatory chromatography column

Sample was eluted from the column

Sample was evaporated to dryness

Sample was reconstituted in an appropriate chromatography buffer

Sample. was analyzed by liquid chromatography-tandem mass spectrometry (LC-MS-MS) as follows:

Sample peptides were fragmented by collisional activation

Characteristic peptide fragments at m/z 301; 471 and 568 were identified as predominant fragments

Peptides that fragmented to m/z 301; 471 and 568 were identified as post-translational modifications of Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:3), wherein the Pro at positions 14 and 26 are 4-hydroxyproline, as discussed below.

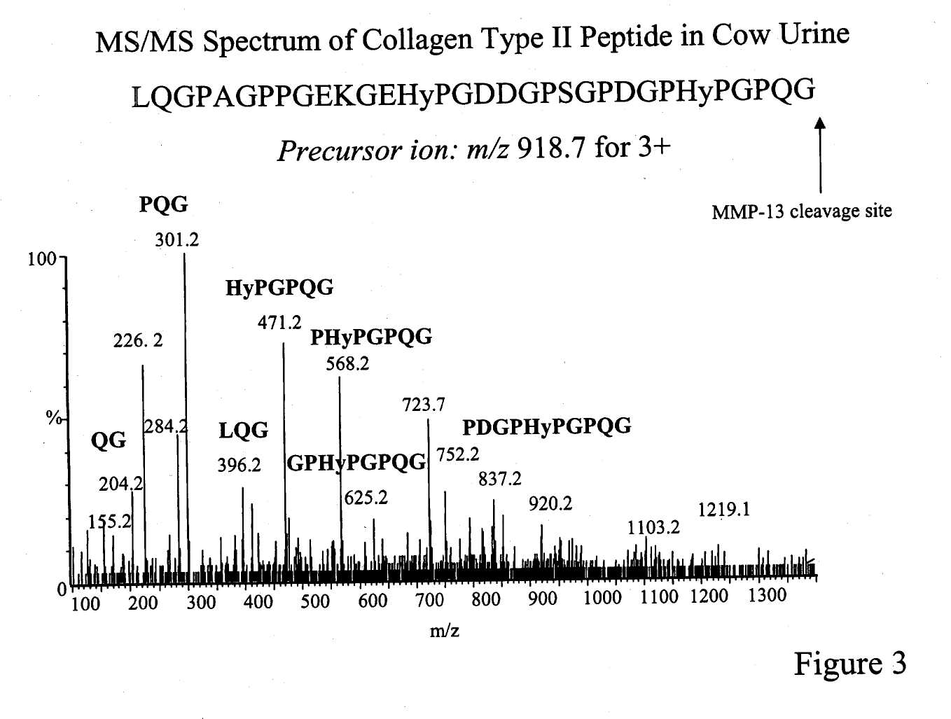

A fragmentation of a collagen type II peptide (SEQ ID NO:3) having the Gly-Pro-Pro-Gly-Pro-Gln-Gly (SEQ ID NO: 1) C-terminus wherein the proline at position 3 is hydroxylated is illustrated in FIG. 3. The vertical (Y-axis) represents the fragment ion abundance (intensity) detected for a specific mass to charge ratio expressed as a percent of the total ions detected. The horizontal (X-axis) represents the mass to charge ratio of the detected fragment ions. The predominant fragmentation products of the peptides in the urine sample were identified as m/z 301; 471 and 568.

Software was used to match the fragment ions in an MS/MS spectrum of the peptide to the theoretical sequence ions produced in silico by cleaving all proteins in a public database at every peptide bond (no enzyme specificity). The software matched the observed and theoretical molecular weights of an intact peptide and its fragment ions based on expected cleavages of the peptide between each of the amino acids. The software was employed to match the observed data to all possible peptides derived from all the proteins in a global protein database of known mammalian proteins. The program determined that the peptide having the mass m/z 918.7 for 3+, which fragments to yield ions at m/z 301; 471 and 568 was the post-translational modification of Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:3), wherein the proline at positions 14 and 26 are 4-hydroxyproline.

To prove that the software determined the correct peptide, a standard of the peptide was synthesized and its LC retention time and MS/MS spectrum was matched to that of the peptide found in urine. The analytical data matched.

### EXAMPLE 3

### Identification and Quantification of Human Collagen Type II Peptide Fragment

The procedures used in Example 2 are used to identify the presence of peptide SEQ ID NO: 2 in urine samples obtained from a human subject.

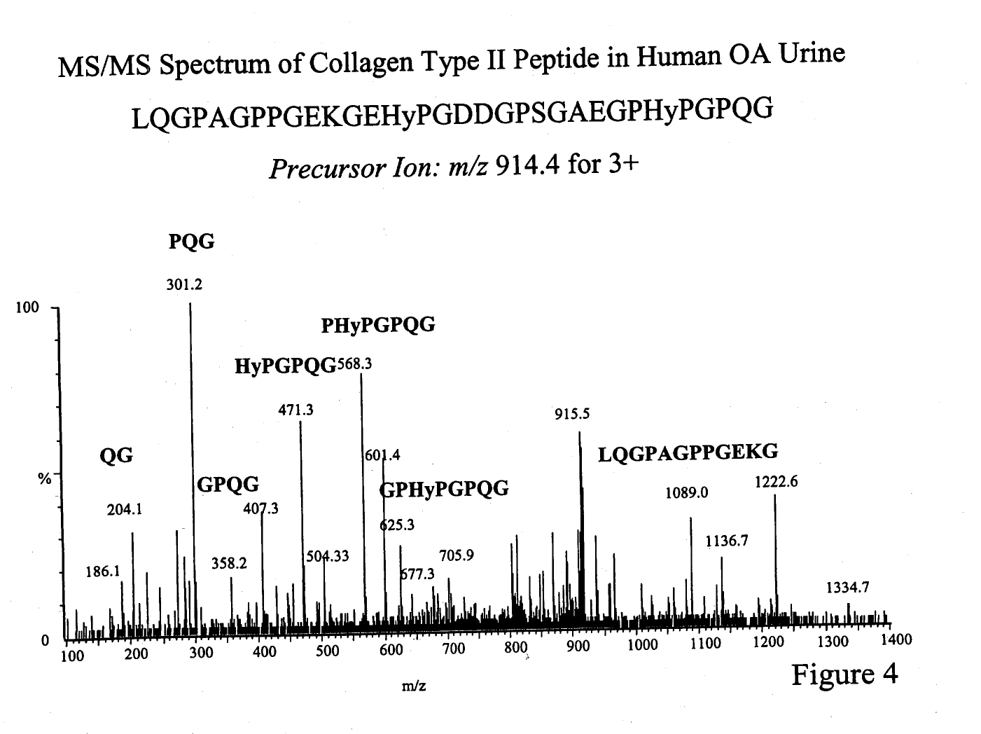

FIG. 4 shows the fragmentation of a peptide in the sample from a human subject. As shown, the peptide is the post-translationally modified peptide Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2) wherein prolines at positions 14 and 26 are hydroxylated to 4-hydroxyproline. The peptide SEQ ID NO:2 has the identified mass/change of 914.4 with a charge of 3+. Fragmentation yielded the characteristic product ions of 301, 471, and 568 corresponding to SEQ. ID NO: 5, SEQ ID NO:6, and SEQ ID NO:7, respectively.

The sample is compared to a standard value representing a concentration of peptide corresponding to SEQ ID NO 2 found in a human of similar age, sex, and ethnic background for evaluation of deviation from the standard value. The deviation from the standard value forms the basis of diagnosis and treatment for a sample value deviating significantly from the standard value, in a fashion similar to the method described in Example 1.

### EXAMPLE 4

### Identification and Quantification of Collagen Type II in Dog Urine Samples

The procedures used in Example 2 were used to identify the presence of peptide Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:4), wherein the proline at position 26 is 4-hydroxyproline in urine samples obtained from canine subjects of a breed known to have a predilection for canine hip dysplasia.

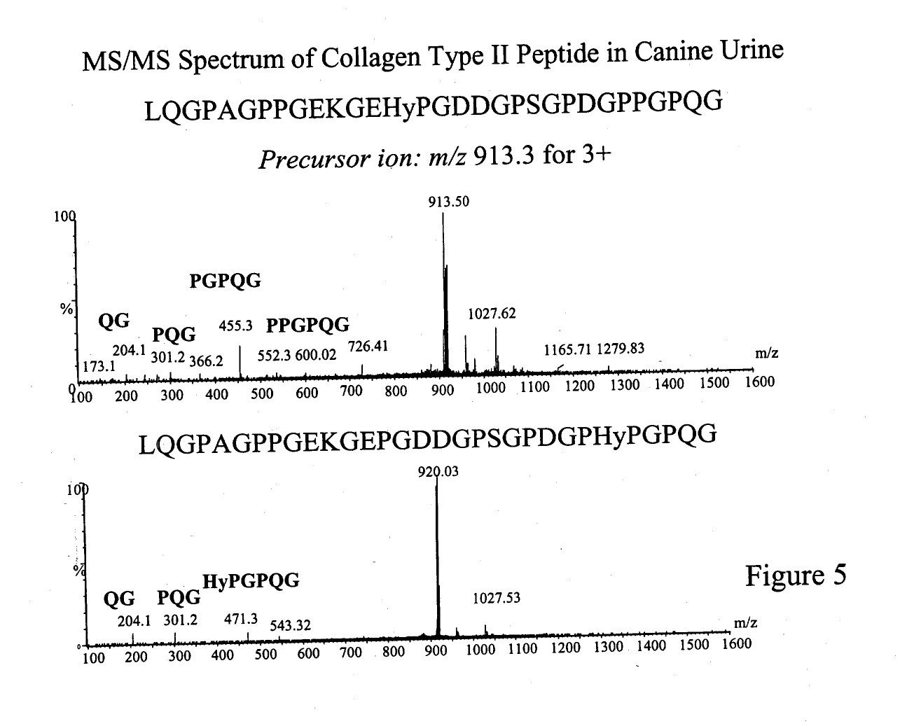

FIG. 5 shows the fragmentation of a peptide in the sample. As shown, the peptide is the post-translational modification of peptide Leu-Gln-Gly-Pro-Ala-Gly-Pro-Pro-Gly-Glu-Lys-Gly-Glu-Pro-Gly-Asp-Asp-Gly-Pro-Ser-Gly-Pro-Asp-Gly-Pro-Pro-Gly-Pro-Gln-Gly (SEQ ID NO:4), wherein the proline at position 14 or 26 is hydroxylated to 4-hydroxyproline. The peptide SEQ ID NO:4 has the mass/charge of 913.4 with a charge of 3+. Fragmentation yielded the characteristic product ions at m/z 301, 471 or 455, and 568 or 552, corresponding to SEQ. ID NO:5, SEQ ID NO:6, and SEQ ID NO:7, respectively. The same peptide sequence is present in at least two post-translationally-modified forms in canine urine: one form where proline-14 is hydroxylated as shown in the top tandem mass spectrum and a second form where proline-26 is hydroxylated as shown in the bottom tandem mass spectrum.

The concentration of peptide corresponding to SEQ ID NO 5 or SEQ ID NO 6 is compared to that of a standard value corresponding to dogs of similar age, breed and sex having a normal growth velocity. If the sample concentration significantly exceeds that of the normal standard, an appropriate veterinary growth inhibiting drug or agent may be administered to ameliorate the potential for the subject dog to acquire, or exhibit advanced symptoms of, hip dysplasia.

Peptides of SEQ. ID NO 1, SEQ ID NO 2 and SEQ ID NO 3 can include modifications of proline to 4 hydroxyproline at positions 8, 14 and 26 and lysine to 4 hydroxylysine at position 11. It will be noted that independent of the species of animal, upon fragmentation the predominant peptide yielded fragment ions having mass to charge ratios of approximately 301, 471 and 568. Fragmentation of the originally transcribed peptide (without post-translational conversion of the proline at position 26 to 4-hydroxyproline) would yield characteristic fragment ions having mass to charge ratios of approximately 301, 455 and 552 and are included within the scope of collagen degradation products.

Other characteristic fragment ions may be diagnostic within a given species. By way of example, FIG. 3 indicates that characteristic fragment ions having mass to charge ratios of 396 and 1219 are produced upon fragmentation of peptide SEQ ID NO: 3, found in samples from cattle. Also, as an example, FIG. 4 indicates that fragment ions having mass to charge ratios of 407 and 1222 are produced upon fragmentation of peptide SEQ ID NO: 2, found in samples from humans. Although the fragment ions having mass to charge ratios of approximately 301, 471 and 568 represent a preferred embodiment of the invention, other specific fragment ions may have utility when analyzing samples from that species and are included within the scope of collagen degradation products.

It is predicted that peptide SEQ ID NO: 8 will be found in specimens from rats, peptide SEQ ID NO:9 will be found in specimens from rabbits, SEQ ID NO: 10 will be found in specimens from mice; and peptide SEQ ID NO: 11 will be found in specimens from horses as well as post-translational modifications of these peptides when these species exhibit signs and symptoms of a growth disorder due to abnormal collagen degradation, and likely even prior to manifestation of the signs and symptoms of a growth disorder.

Since it is now known that a specific peptide, for example, Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2), has been identified as the predominant collagen type II degradation peptide in the biological fluid sample of humans, the relative quantity of that peptide in a given sample provides an indication of the extent of collagen II degradation occurring in the subject.

The peptide SEQ ID NO:2, SEQ ID NO:3, or SEQ ID NO:4 and likely SEQ ID No:8, SEQ ID NO:9, SEQ ID NO:10, SEQ ID NO:11 can be quantified by the following general, illustrative procedure:

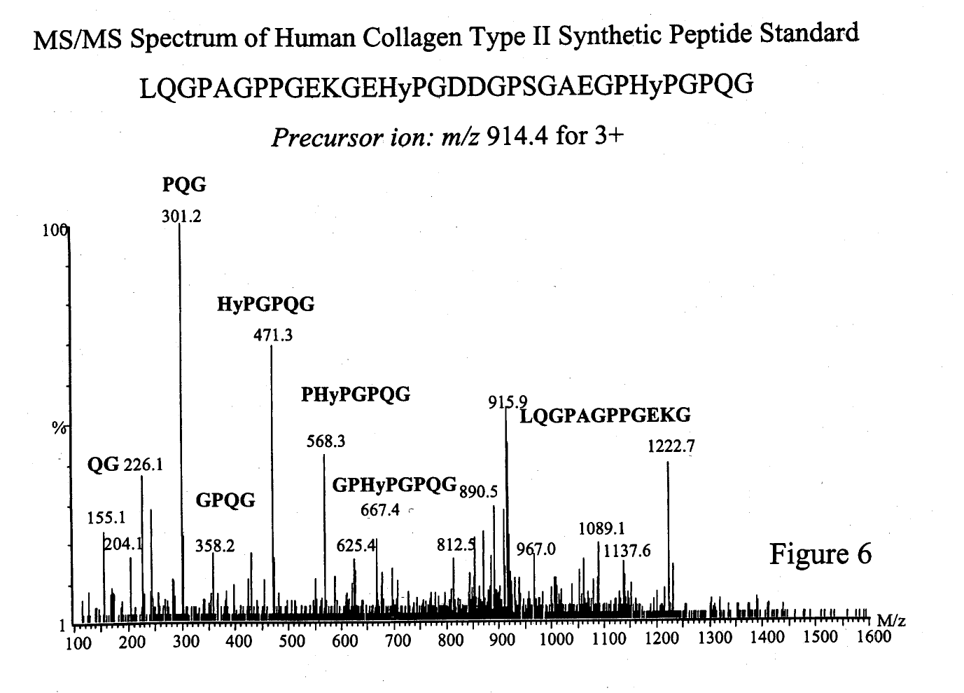

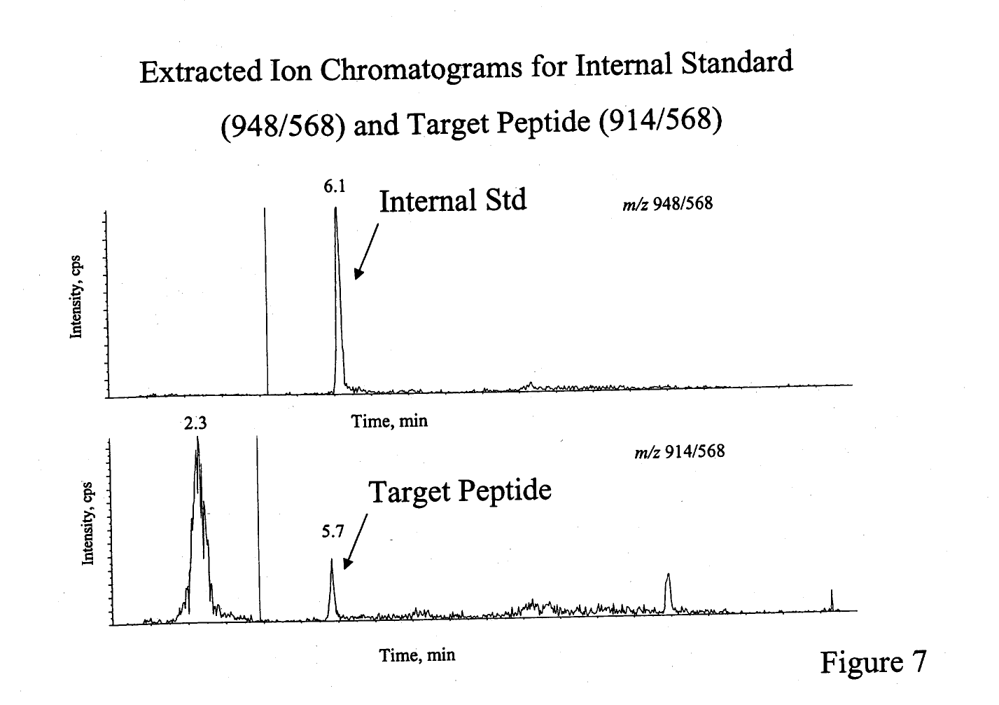

A biological specimen, for example urine, is collected from the subject. The peptide, which can be referred to as the subject or target peptide, having the appropriate mass, for example m/z 918.7 for (M+3H)3+ (SEQ ID NO: 3) is extracted from the sample using procedures known to the art. See Example 4, below. The sample is spiked with a known quantity of a synthetic peptide (known as the internal standard) having a sequence very similar to the target peptide sequence. For example, peptide Val-Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO. 14), may be used as an internal standard. The sample is prepared and introduced to the LC-MS-MS for analysis. The presence of the target peptides is confirmed by three criteria: 1) it elutes from the LC column at the proper time, 2) it is the correct molecular weight, and, 3) upon collisional activation, yields the characteristic fragments of approximately m/z 301, 471 and 568 (shown in FIG. 6), or other known fragment ions of the peptide. FIG. 7 is a chromatogram showing the elution profile of the target peptide and the internal standard as a representative example. Comparison of the abundance of the target peptide (area under peak) to the abundance of a standard, normalized by the abundance of an internal standard indicates a relative quantity of target peptide in the sample. More particularly, the area under the curve representing the abundance of the standard peptide is compared to the area under the curve representing the abundance of the target peptide to obtain a relative quantity of target peptide in a sample. The areas of the standard and target peptides are normalized relative to the area of an internal standard peptide to adjust for sample-to-sample variability in extraction efficiency, detection, and other potential variables. Of course, acceptable methods of quantifying the target peptide in a biological sample are encompassed by the scope of the invention. A general quantitation procedure for collagen type II peptide biomarker in human urine is set out below in Example 5 as a representative procedure.

### EXAMPLE 5

### General Quantitation Procedure For Collagen Type II Peptide Biomarker In Human Urine

1. A suitable population of morphologically normal, age-appropriate height and weight subjects is selected for obtaining control samples. First pass urine is collected in polypropylene tubes. Samples are frozen at −80C until sample workup.

2. 100 microliters of urine is aliquoted for creatinine quantitation, which is performed using standard procedures known in the art

3. Standard solutions ranging from 30 pg/mL to 100 ng/mL are prepared using a synthetic peptide standard

4. An internal standard (similar peptide) is spiked into 30 mL

of each urine sample and standard to a final concentration of 1.0 nM

5. Urine solutions are extracted by mixed-phase (RP and AEX) preparatory chromatography:

a. Equilibrate cartridge with 10 mL of MeOH

b. Condition cartridge with 10 mL of 50 mM NH40Ac, pH 7

c. Load Sample (pH 7)

d. Wash with 10 mL of 50 mM NH40Ac, pH 7

e. Wash with 10 mL 5% MeOH

f Elute with 1 mL 5% Formic Acid, 95% MeOH

6. Eluant is evaporated to dryness and reconstituted in 100 microliters of 2% formic acid solution

7. Solutions are analyzed by LC/MS/MS

8. The collagen peptide in samples is quantified by correlating LC/MS/MS responses to those of the standards, normalized for the internal standard responses

9. Final peptide concentrations are normalized to creatinine levels

### Preparation of Standard Curve

1. Prepare Stock Solutions of Collagen II Peptide:

10 ng/mL, 100 ng/mL, 1 μg/mL, and 10 μg/mL.

2. Prepare Stock Solution of Internal Standard: 3 μg/mL

3. Add 100 μL of Internal Standard (3 μg/mL) to 30 mL of urine

4. Add 300 μL of 10 ng/mL, 100 ng/mL, 1 μg/mL, and 10 μg/mL to 30 mL of urine to get 100 pg/mL, 1 ng/mL, 10 ng/mL, and 100 ng/mL standards, respectively. To get 30 pg/mL standard add 90 μL of 10 ng/mL stock solution to 30 mL of urine.

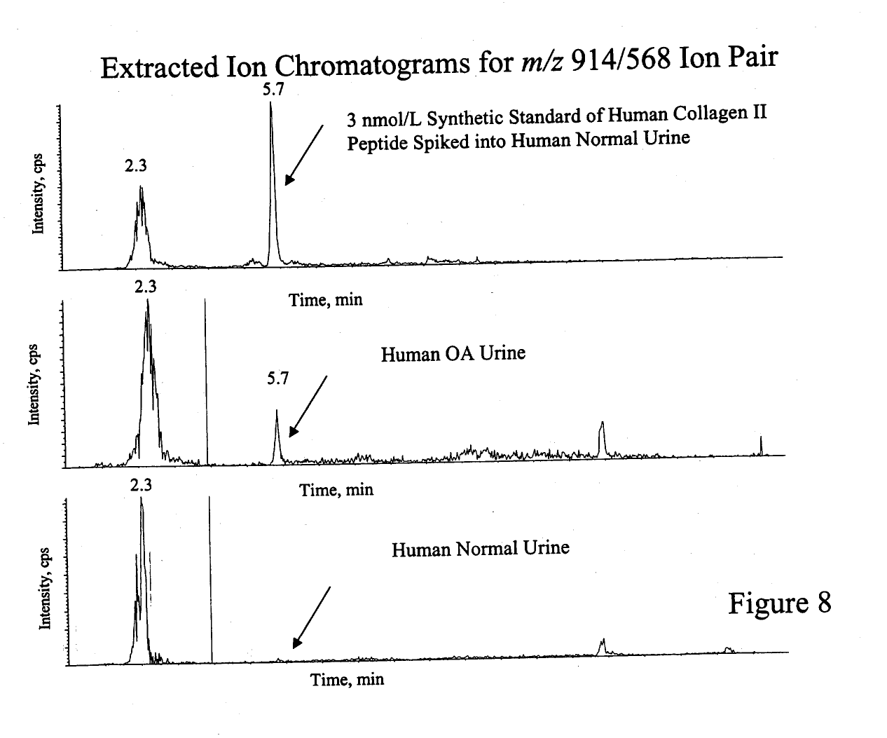

FIG. 7 graphically illustrates a comparison of extracted ion chromatograms for the 914/568 Ion Pair, which was used to measure the collagen type II peptide (SEQ ID NO: 2). The bottom scan shows that the peptide eluting at 5.7 minutes was not detected in human urine of a subject without signs and symptoms of a growth disorder. The middle scan shows detectable levels of the peptide, and the top scan shows that the synthetic analog of this peptide elutes at the same time. FIG. 8 illustrates a standard curve of the collagen type II peptide spiked into control human urine at various concentrations ranging from 30 pg/mL to 100 ng/mL. The response is linear over this concentration range for accurate quantification.

This quantification of the peptides can be used for diagnosis or prognosis of diseases such as acromegaly and short stature, to monitor collagenase enzyme activity in disease or physiological conditions characterized by abnormal collagenase activity, and to evaluate drugs or agents used to modulate growth. Consequently, the peptides SEQ ID NO:2, SEQ I) NO:3 or SEQ ID NO:4 in biological samples are biomarkers of disease or conditions in which abnormal collagen type II proteolysis is characteristic. The identification and quantification of thebiomarker peptides can beusedin diagnosis and prognosis of diseases or conditions characterized by abnormal collagen II degradation.

Further, the identification and quantification of the biomarkers peptides SEQ ID NO:2, SEQ ID NO:3 or SEQ ID NO:4, for example, in a biological sample can be used to monitor or evaluate the efficacy of a drug or other agent used to block the activity and/or abundances of the proteolytic enzyme(s) that yields the degradation products, peptides SEQ ID NO:2, SEQ ID NO:3 or SEQ ID NO:4. One exemplary use of the biomarker of the present invention is to monitor and evaluate the activity of human growth hormone. The identification and quantification of peptides SEQ ID NO:2, SEQ ID NO:3 or SEQ ID NO:4 or post-translational modifications, in biological fluid samples can indicate collagen type II degradation and the relative in vivo activity of a collagenase enzyme, for example MMP-13. In diseases or physiologic conditions in which abnormal collagen type II degradation is a pathological characteristic, administration of a pharmacologically effective amount of human growth hormone, for example, should result in the normalization of the concentration of peptide Leu-Gln-Gly-Pro-Ala-Gly-Pro-Pro-Gly-Glu-Lys-Gly-Glu-Pro-Gly-Asp-Asp-Gly-Pro-Ser-Gly-Ala-Glu-Gly-Pro-Pro-Gly-Pro-Gln-Gly (SEQ ID NO:2), for example, in a human subject's biological fluid sample.

Determination of the biomarker concentration over time can indicate a progression or regression of disease. Changes in the biomarker concentration can be used to evaluate the effectiveness of treatments. For example, successful treatment of the subject with human growth hormone would result in a increase in the relative level of collagen type II peptide in a biological sample taken from the patient. It will be appreciated that there may be other methods of determining the concentrations of collagen degradation products in a biological sample, and that these methods are within the scope of the present invention.

It will be appreciated by those skilled in the art that characteristic and identifiable derivatives or modifications of the novel biomarkers of the present invention can be produced using recognized techniques. For example, peptides SEQ ID NO: 2 SEQ ID NO: 3 or SEQ ID NO: 4 or their post-translational analogs could be enzymatically or chemically cleaved to yield one or more derivatives that would be readily identifiable. Such derivatives consequently could function as a biomarker. Likewise, for example, peptides SEQ ID NO: 2 SEQ ID NO: 3 or SEQ ID NO: 4 or their post-translational analogs could be modified, perhaps by changing substituting, adding or deleting one or more amino acid residues, resulting in an identifiable modified peptide that could function as a biomarker. It will be appreciated, therefore, that any derivative or modification of the marker peptides of the present invention represent further embodiments of the invention and fall within the scope of the appended claims.

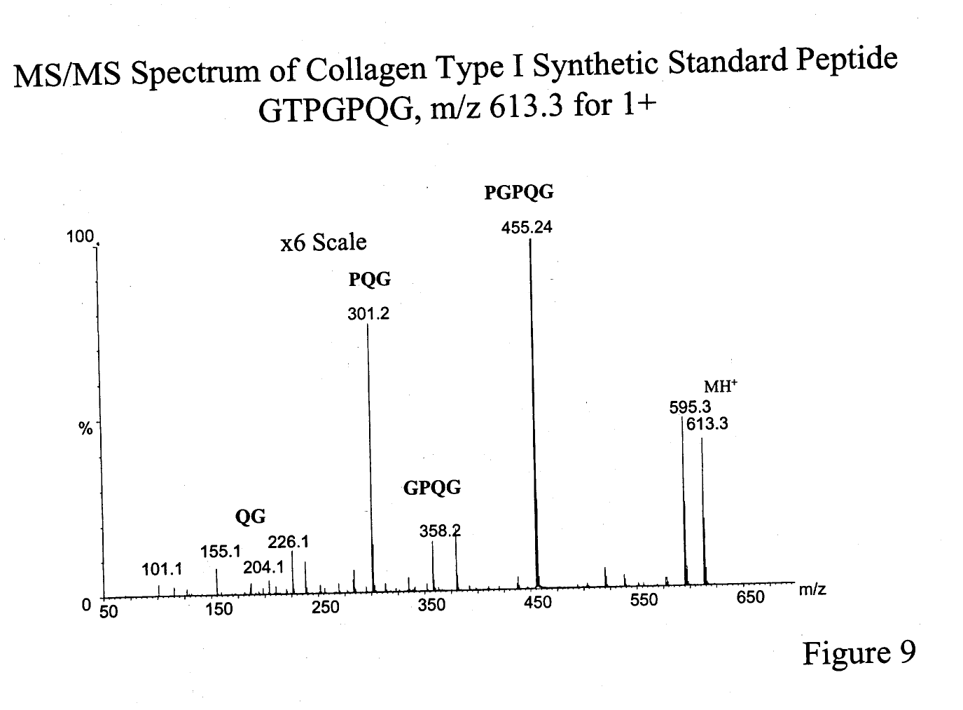

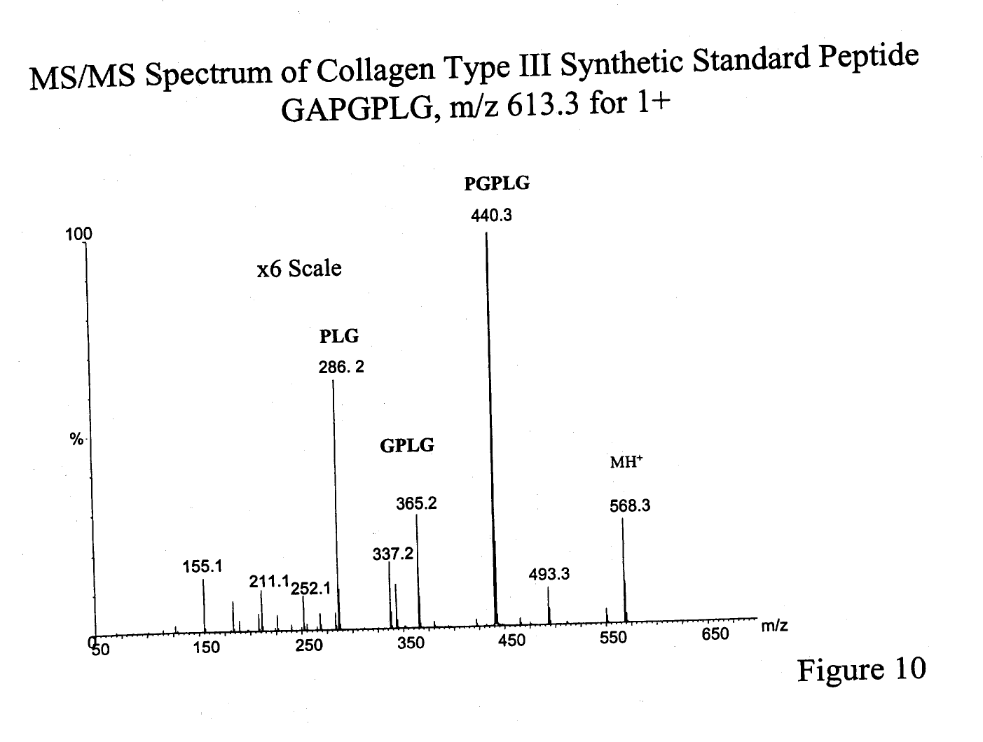

The analytic principles of the present invention also can be used to identify and quantify peptides which may serve as biomarkers of breakdown of collagens other than collagen type II. The peptide sequence Gly-Thr-Pro-Gly-Pro-Gln-Gly (SEQ ID NO: 12) is the expected C-terminus sequence of a biomarker peptide resulting from metalloproteinase cleavage of collagen type I, based upon an analysis of the peptide database. FIG. 9 illustrates the characteristic fragment ions resulting from the collisional activation of synthesized SEQ ID NO: 12. Collagen type I breakdown peptides can be identified in a biological sample using the methods of the present invention based upon the identification of characteristic fragment ions of m/z 301 and 455 for the non-hydroxylated form and m/z 301 and 471 for the hydroxylated form. Likewise, FIG. 10 illustrates that the peptide C-terminal sequence Gly-Ala-Pro-Gly-Pro-Leu-Gly (SEQ ID NO: 13) expected from the enzymatic cleavage of collagen type III will also yield identifiable, characteristic fragment ions when analyzed by methods of the present invention. The collagen type m fingerprint fragment ions are m/z 286 and 440 for the non-hydroxylated form and would be m/z 286 and 471 for the hydroxylated form. FIGS. 9 and 10 not only identify characteristic fragment ions that can be used to identify breakdown peptides specifically from collagen types I and III, respectively, but also substantiate the fact that the methods of the present invention can be employed more broadly to identify characteristic cleavage products of any collagen.

Although the previously described method of identification and quantification is one preferred method of identifying and quantifying peptides SEQ ID NO:2, SEQ ID NO: 3 or SEQ ID NO: 4, for example, in biological samples, the identification and quantification of these peptides by other assays is contemplated by the scope of the invention. Because the peptides SEQ ID NO: 2, SEQ ID NO: 3 and SEQ ID NO: 4 have now been identified, analytical tools other than mass spectrometry or tandem mass spectrometry may be employed to determine the presence of these specific degradation proteins. For example, known ultraviolet spectrometry, electrospray ionization, electrochemical or flame-ionization methods may be employed without departing from the scope of the invention. Known sequencing methods are also included within the scope of the invention. Also, enzyme-linked immunoassay (ELISA) or other radioimmunoassay (RIA) techniques that specifically recognize the described peptide sequences or post-translational modifications thereof, may be employed. Specifically, a neoepitope antibody to the N-terminus of the peptide may provide greater sensitivity and specificity than current ELISA methods that use a C-terminal neoepitope antibody and a capture antibody to a portion of the sequence. In addition, analytical quantification methods applied to derivatives, digests, or other chemical or enzymatic products of the target peptide or its post-translational modifications thereof, are covered by the scope of this invention.

Although the illustrative examples involved the identification and quantification of the peptides in urine samples, the peptides may be detected in other biological fluid samples or extracts, such as blood, plasma, spinal fluid, amniotic fluid, or synovial fluid. Also, samples of tissues, such as tissue around a joint, may be obtained by biopsy or the like, and the peptides isolated through techniques known to the art. Identification and quantification of the biomarkers carried out by methods of the present invention then may be employed to identify and quantify the biomarkers in the tissue sample.

Deviation from a normal concentration of collagen degradation product (that is to say, the concentration of collagen degradation products obtained from individuals who do not exhibit the signs or symptoms of a growth disorder) may be determined in a variety of ways. For example, a concentration that falls anywhere outside of the standard deviation of a median sample may be considered to indicate a growth disorder. Alternatively, an absolute value greater than ten percent outside of the standard deviation may be used to indicate a growth disorder. In another alternative, an absolute value greater than fifteen percent outside of the standard deviation may be used to indicate a growth disorder. In yet another alternative, an absolute value greater than twenty percent outside of the standard deviation may be used to indicate a growth disorder. In still another alternative, an absolute value greater than twenty-five percent outside of the standard deviation may be used to indicate a growth disorder. In another alternative, an absolute value greater than fifty percent outside of the standard deviation may be used to indicate a growth disorder. In still another alternative, an absolute value greater than seventy percent outside of the standard deviation may be used to indicate a growth disorder. In yet another alternative, an absolute value greater than twice the standard deviation may be used to indicate a growth disorder. In still another alternative, an absolute value greater than a range between about ten percent outside of the standard deviation and about ten times the standard deviation may be used to indicate a growth disorder. In yet another alternative, an absolute value greater than a range between about ten percent outside of the standard deviation and about five times the standard deviation may be used to indicate a growth disorder. In still another alternative, an absolute value greater than a range between about ten percent outside of the standard deviation and about three times the standard deviation may be used to indicate a growth disorder. In still another alternative, an absolute value greater than a range between about ten percent outside of the standard deviation and twice the standard deviation may be used to indicate a growth disorder.

In yet another alternative, an absolute value greater than a range between about ten percent outside of the standard median value and about twenty times the standard median value maybe used to indicate a growth disorder. In still another alternative, an absolute value greater than a range between about ten percent outside of the standard median value and about ten times the standard median value may be used to indicate a growth disorder. In yet another alternative, an absolute value greater than a range between about ten percent outside of the standard median value and about five times the standard median value may be used to indicate a growth disorder. In still another alternative, an absolute value greater than a range between about ten percent outside of the standard median value and about three times the standard median value may be used to indicate a growth disorder. In still another alternative, an absolute value greater than a range between about ten percent outside of the standard median value and about twice the standard median value may be used to indicate a growth disorder.

In yet another alternative, an absolute value greater than a range between about ten percent outside of the standard mean value and about twenty times the standard mean value may be used to indicate a growth disorder. In still another alternative, an absolute value greater than a range between about ten percent outside of the standard mean value and about ten times the standard mean value may be used to indicate a growth disorder. In yet another alternative, an absolute value greater than a range between about ten percent outside of the standard mean value and about five times the standard mean value may be used to indicate a growth disorder. In still another alternative, an absolute value greater than a range between about ten percent outside of the standard mean value and about three times the standard mean value may be used to indicate a growth disorder. In yet another alternative, an absolute value greater than a range between about ten percent outside of the standard mean value and about twice the standard mean value may be used to indicate a growth disorder. Those skilled in the art will recognize that other statistical measurements of variation may be employed in determining a value that lies outside of the standard normal value for the determination of an abnormal concentration of collagen degradation products. These methods are within the scope of the present invention and the appended claims

What is claimed is:

1. A method of determining an appropriate level of collagen degradation products in a mammal comprising:
obtaining at least one biological sample from each member of a defined population of morphologically normal, age-appropriate height and weight subjects;
determining the concentration of at least one collagen degradation product for each subject within said defined population within said sample;
recording said determined concentration as a numerical value representing said concentration;
recording at least on attribute of said subject within said population selected from age, weight, sex, ethnic origin, or a combination thereof; and
defining a statistical correlation based upon said attribute and said concentration.

2. The method of claim 1 wherein said mammal is human.

3. The method of claim 1 wherein said mammal is bovine.

4. The method of claim 1 wherein said mammal is canine.

5. The method of claim 1 wherein said biological sample is selected from urine, blood, plasma, synovial fluid, and amniotic fluid.

6. The method of claim 5 wherein said biological sample is urine.

7. The method of claim 1 wherein said collagen degradation product is a peptide corresponding in amino acid sequence to SEQ ID NO 2.

8. The method of claim 1 wherein said collagen degradation product is a peptide corresponding in amino acid sequence to SEQ ID NO 1.

9. The method of claim 1 wherein said collagen degradation product is a peptide corresponding in amino acid sequence to SEQ ID NO 3.

10. The method of claim 1 wherein said collagen degradation product is a peptide corresponding in amino acid sequence to SEQ ID NO 4.

11. The method of claim 1 wherein said collagen degradation product is a peptide corresponding in amino acid sequence to SEQ ID NO 5.

12. The method of claim 1 wherein said collagen degradation product is a peptide corresponding in amino acid sequence to SEQ ID NO 6.

13. The method of claim 1 wherein said collagen degradation product is a peptide corresponding in amino acid sequence to SEQ ID NO 7.

14. The method of claim 1 wherein said collagen degradation product is a peptide corresponding in amino acid sequence to SEQ ID NO 8.

15. The method of claim 1 wherein said collagen degradation product is a peptide corresponding in amino acid sequence to SEQ ID NO 9.

16. The method of claim 1 wherein said collagen degradation product is a peptide corresponding in amino acid sequence to SEQ ID NO 10.

17. The method of claim 1 wherein said collagen degradation product is a peptide corresponding in amino acid sequence to SEQ ID NO 11.

18. The method of claim 1 wherein said collagen degradation product is a peptide corresponding in amino acid sequence to SEQ ID NO 12.

19. The method of claim 1 wherein said collagen degradation product is a peptide corresponding in amino acid sequence to SEQ ID NO 13.

20. The method of claim 1 wherein said collagen degradation product is a peptide corresponding in amino acid sequence to SEQ ID NO 14.

21. The method of claim 1 wherein the determination of said concentration of said sample is performed with enzyme-linked immunoassay.

22. The method of claim 1 wherein the determination of said concentration of said sample is performed with mass spectroscopy.

23. The method of claim 22 wherein said mass spectroscopy is tandem mass spectroscopy.

24. A kit comprising at least one reagent suitable for detecting the presence of a polypeptide having the sequence as set forth in SEQ ID 1, 2, or 3 in a biological sample.

25. The kit of claim 24, wherein the kit comprises an antibody directed against a polypeptide having the sequence as set forth in SEQ ID NO:1, SEQ ID NO:2, or SEQ ID NO:3.

26. The kit of claim 25, wherein the antibody is a polyclonal antibody.

27. The kit of claim 26, wherein the polyclonal antibody is an affinity-purified poyclonal antibody.

28. The kit of claim 25, wherein the antibody is a monoclonal antibody.

29. The kit of claim 28, wherein the monoclonal antibody is of a class selected from the group consisting of IgG, IgA, IgD, IgE, and IgM.

30. An antibody which specifically binds a peptide consisting of a sequence as set forth in SEQ ID NO:1, SEQ ID NO:2, or SEQ ID NO:3.

31. The antibody of claim 30, wherein the antibody is a polyclonal antibody.

32. The antibody of claim 31, wherein the polyclonal antibody is an affinity-purified polyclonal antibody.

33. The antibody of claim 30, wherein the antibody is a monoclonal antibody.

34. The antibody of claim 33, wherein the monoclonal antibody antibody is of a class selected from the group consisting of IgG, IgA, IgD, IgE, and IgM.

35. A method of detecting a disease or condition involving an abnormal levels of one or more collagen fragments, the method comprising assessing the presence and/or amount of at least one collagen fragment which comprises a sequence as set forth in SEQ ID NO:1.

36. The method of claim 35, wherein the at least one collagen fragment consists of a polypeptide having a sequence as set forth in SEQ ID NO:2.

37. The method of claim 37, wherein the at least one collagen fragment consists of a polypeptide having a sequence as set forth in SEQ ID NO:3.

38. A method of identifying a compound which modulates the presence and amount of at least one collagen fragment in a mammal, the method comprising administering a test compound to a mammal and assessing the presence and/or amount of at least one collagen fragment which comprises the sequence as set forth in SEQ ID NO:1.

39. The method of claim 38, wherein the at least one collagen fragment consists of a polypeptide having a sequence as set forth in SEQ ID NO:2.

40. The method of claim 39, wherein the at least one collagen fragment consists of a polypeptide having a sequence as set forth in SEQ ID NO:3.

41. A method of monitoring efficacy of an agent administered to a mammal to treat a disorder involving abnormal levels of collagen degradation, the method comprising assessing the presence and/or amount of at least one collagen fragment which comprises a sequence as set forth in SEQ ID NO: 1.

42. The method of claim 41, wherein the at least one collagen fragment consists of a polypeptide having a sequence as set forth in SEQ ID NO: 2.

43. The method of claim 41, wherein the at least one collagen fragment consists of a polypeptide having a sequence as set forth in SEQ ID NO: 3.

44. A method of diagnosing an individual suspected of having a disorder involving an abnormal level of collagen degradation compared to individuals not having the disease, the method comprising assessing the presence and/or amount of at least one collagen fragment which comprises a sequence as set forth in SEQ ID NO:1.

45. The method of claim 44, wherein the at least one collagen fragment consists of the sequence as set forth in SEQ ID NO:2.

46. The method of claim 45, wherein the at least one collagen fragment consists of the sequence as set forth in SEQ ID NO: 3.

47. The method of claim 44, wherein the individuals not having the disorder are in a matched group with the individual.

48. The method of claim 47, wherein the group is matched according to criteria selected from the group consisting of age, gender, weight, and medical history.

49. The method of claim 48, wherein the amount of collagen degradation product is at least 25% different from that in individuals not having the disorder.

50. The method of claim 49, wherein the amount of collagen degradation product is at least 50% different from that in individuals not having the disorder.

51. The method of claim 44, wherein the amount of collagen degradation product is at least 100% different from amount of collagen degradation product of an unmatched population not having the disorder.

52. The method of claim 44, wherein the amount of collagen degradation product is at least 200% different from amount of collagen degradation product of an unmatched population not having the disorder.

53. The method of claim 44, wherein the disorder is characterized by short stature.

54. The method of claim 53, wherein the disorder is selected from the group consisting of intrauterine growth retardation, skeletal dysplasia, neurofibromatotsis, Cushing's syndrome, hypothyroidism, panhypopituitarism, Turner syndrome, Noonan syndrome, Russell-Silver syndrome, Williams syndrome, and pseudohypothyroidism and short stature.

55. The method of claim 44, wherein the disorder is characterized by tall stature.

56. The method of claim 55, wherein the disorder is selected from the group consisting of Klinefelter syndrome, thyrotoxosis, glucocorticoid resistance, and acromegaly.

57. The method of claim 44, further comprising assessing the presence and/or amount of at least one growth-modulating compound.

58. The method of claim 57, wherein the at least one growth-modulating compound is selected from the group consisting of human growth hormone, insulin-like growth factor-1, Pegvisomant, Trovert, and Somavert.

59. The method of claim 58, wherein the assessing the presence and/or amount of at least one growth-modulating compound comprises detecting the compound by an immunological assay.

60. The method of claim 59, wherein the immunological assay is selected from the group consisting of ELISA, radioimmunoassay, immunoprecipitation, Western blotting, electrochemiluminescence, and immunodiffusion.

61. The method of claim 57, wherein the assessing the presence and/or amount of at least one growth-modulating compound comprises detecting the compound using mass spectroscopy.

62. A method of identifying naturally occurring collagen fragments, the method comprising detecting polypeptide fragments comprising a sequence as set forth in SEQ ID NO:1.

63. The method of claim 62, wherein the collagen fragment comprising a sequence as set forth in SEQ ID NO:1 comprises a sequence as set forth in SEQ ID NO:2.

64. The method of claim 62, wherein the collagen fragment comprising a sequence as set forth in SEQ ID NO:1 comprises a sequence as set forth in SEQ ID NO:3.

65. A method of diagnosing an individual suspected of having a disorder involving abnormal growth, the method comprising assessing the presence and/or amount of at least one collagen fragment which comprises a sequence as set forth in SEQ ID NO:1.

66. The method of claim 65, further comprising assessing the presence and/or amount of at least one hormone which regulates growth.

67. The method of claim 66, wherein the at least one hormone is selected from the group consisting of growth hormone and IGF1.

68. The method of claim 65, wherein the at least one collagen fragment consists of a sequence as set forth in SEQ ID NO:2.

69. The method of claim 65, wherein the at least one collagen fragment consists of a sequence as set forth in SEQ ID NO:3.

70. The method of claim 65, wherein the disorder is characterized by short stature.

71. The method of claim 70, wherein the disorder is selected from the group consisting of intrauterine growth retardation, skeletal dysplasia, neurofibromatotsis, Cushing's syndrome, hypothyroidism, panhypopituitarism, Turner syndrome, Noonan syndrome, Russell-Silver syndrome, Williams syndrome, and pseudohypothyroidism and short stature.

72. The method of claim 65, wherein the disorder is characterized by tall stature.

73. The method of claim 72, wherein the disorder is selected from the group consisting of Klinefelter syndrome, thyrotoxosis, glucocorticoid resistance, and acromegaly.

74. A method of diagnosing a growth disorder in a mammal comprising:
obtaining at least one biological sample from a subject;
measuring the concentration of a collagen degradation product in said sample;
comparing said measured concentration of said collagen degradation product with a standard value, said standard value comprising a statistical representation of a mammal of at least the same species, sex, and age; and
determining the presence of a growth disorder when said sample concentration exceeds a threshold value outside of said standard value.
